(19) United States
(12) Patent Application Publication
(10) Pub. No.: US 2025/0312584 A1
(43) Pub. Date: Oct. 9, 2025

(54) METHOD FOR MANUFACTURING POLYMER-BASED MICRONEEDLE PATCH AND MICRONEEDLE PATCH MANUFACTURED THEREBY

(71) Applicant: Daegu Gyeongbuk Institute of Science and Technology, Daegu (KR)

(72) Inventors: Sang Hoon LEE, Daegu (KR); Jin Woong JEONG, Sejong (KR)

(73) Assignee: Daegu Gyeongbuk Institute of Science and Technology, Daegu (KR)

(21) Appl. No.: 18/881,628

(22) PCT Filed: May 2, 2023
(86) PCT No.: PCT/KR2023/005957

§ 371 Date: Jan. 6, 2025

(30) Foreign Application Priority Data
Oct. 26, 2022 (KR) 10-2022-0139509

(51) Int. Cl. A61M 37/00; B33Y 10/00; B33Y 80/00

(52) U.S. Cl. CPC A61M 37/0015; B33Y 10/00; B33Y 80/00; A61M 2037/0053

(57) ABSTRACT

The present invention relates to a method for manufacturing a polymer-based microneedle patch designed to maximize skin penetration performance. The polymer-based microneedle patch of the present disclosure can be manufactured to have enhanced penetration power and flexibility as microneedles formed of a polymer with both rigidity and flexibility are combined with an elastic and breathable substrate.

### TECHNICAL FIELD

The present invention relates to a method for manufacturing a polymer-based microneedle patch, and more particularly, to a method for manufacturing a polymer-based microneedle patch, based on a microneedle, using a 3D printing process.

### BACKGROUND ART

A microneedle is a technology for penetrating the stratum corneum of skin using a fine needle having a size of several hundred micrometers and delivering a drug to the epidermis and dermis without pain, or for manufacturing an electrode having better performance than a conventional surface electrode. Drug delivery using the microneedle causes less pain and may be conveniently used, as compared with conventional drug delivery using injection. In addition, when compared with an oral preparation, the microneedle may avoid a first-pass effect to maintain a higher bioavailability and allow administration to a desired area, and thus, it may be usefully applied to skin beauty and treatment of various diseases.

The microneedle is being used mainly for drug delivery, but also may be used as a bioelectrode. The most common method used for monitoring bioelectrical signals is collecting electrical signals using an electrode. The electrode may be largely classified into two electrode types: a wet electrode and a dry electrode, depending on the method of attachment to the skin. A representative non-invasive wet electrode is an electrode based on Ag/AgCl which is a universally used commercial electrode.

However, when a signal measurement method using a wet electrode is used, the wet electrode may not reach the conductive layer of the skin due to the presence of stratum corneum interfering with bioelectrical signal measurement, it is difficult to measure a signal accurately, inflammation or allergic reaction by a gel may occur due to a conductive gel, and also, the microneedle may not be used for a long time due to the nature of the gel which hardens over time. In addition, since the stratum corneum of the skin consisting of dead cells should be removed in order to directly use it on the skin, there is a skin damage problem, and it is difficult to measure signals due to the high sensitivity to movement.

It is a dry electrode using the microneedle that was fabricated for securing the limit of a wet electrode. Since the microneedle used in the dry electrode measures signals by penetrating the stratum corneum of skin, it is less affected by the impedance of the stratum corneum of skin, may be worn for a long time by not using a gel, and is in contact with the skin more stably so that it is less affected by movement.

However, the microneedle needs a fabrication process which is highly difficult or complicated. Recently, development of a microneedle which is cheap and more sophisticated and has a complicated form is allowed by a production process using a 3D printing method.

Conventionally, Japanese Patent Laid-Open Publication No. 2019-176146 (MICRONEEDLE PATCH AND METHOD FOR MANUFACTURING MICRONEEDLE PATCH, Sep. 19, 2019) discloses a method for manufacturing a microneedle patch having a needle base material formation process in which printing is performed on a substrate by a 3D printing method to obtain a base material on which a columnar body is formed, and a microneedle formation process in which the base material is immersed in an etching solution to etch the columnar body to form a microneedle.

However, when the microneedle is fabricated by designing, it is different from a structure which is actually designed and printed with a 3D printer, due to the resolution limit of the 3D printer, and thus, the skin penetration performance of the microneedle is deteriorated due to the problem. In addition, the angle of a microneedle bevel is important in skin insertion, and when the microneedle is fabricated using a 3D printer, a cheap and effective process technology for adjusting the angle of the microneedle bevel is needed.

In addition, another limit of the microneedle is the demand of rigidity sufficient to penetrate the skin, and simultaneously also requires flexibility for decreasing transdermal tissue damage and movement artifact after insertion into the skin. Therefore, a method for manufacturing a microneedle using a 3D printer and an etching process in the conventional technology has problems of damage to polymer materials forming the microneedle by the etching solution and complicating the process due to the added etching process.

In addition, when insertion to the skin surface, skin penetration should be performed with minimum rigidity, and after insertion into the skin surface, it is difficult to fabricate the microneedle requiring both rigidity and flexibility due to the resolution limit of the 3D printer by the nature of the microneedle having soft material properties.

### DISCLOSURE

### Technical Problem

An object of the present invention is to provide a microneedle fabricated by controlling an angle of a microneedle tip using a 3D printing process, and a method for manufacturing a polymer-based microneedle patch using the same.

### Technical Solution

In one general aspect, a method for manufacturing a polymer-based microneedle patch includes: microneedle structure fabrication of fabricating a structure in which a plurality of microneedles are arranged, using 3D printing; microneedle mold forming of adding the microneedle structure to a container containing a first polymer material to form a microneedle mold; microneedle array forming of injecting a second polymer material to the microneedle mold and performing curing to form a microneedle array; and microneedle patch manufacturing of arranging the microneedle array on a substrate to manufacture a microneedle patch.

In another general aspect, a microneedle patch manufactured by the method for manufacturing a microneedle patch according to the present invention is provided, wherein a microneedle tip of the microneedle array has an asymmetric structure having a 45 to 55° slope.

In still another general aspect, a method for manufacturing a microneedle structure using 3D printing is provided, wherein the microneedle structure is formed by including a base part and a plurality of microneedles protruding from the base part, and the base part is placed at a tilt angle of 30 to 60° to a stage of a 3D printer.

### Advantageous Effects

According to the present invention, skin penetration performance may be maximized by adjusting a printing tilt angle using a limitation of a printing mechanism of a 3D printer to finely adjust the tip of the microneedle.

In addition, a polymer microneedle having maximized penetration performance may be fabricated by fabricating a microneedle having a finely adjusted microneedle tip printed with a 3D printer as various biocompatible and functional polymer microneedles.

In addition, an optimized microneedle which may be inserted with minimum invasion, minimize skin tissue damage by flexible polymer properties after insertion, and minimize noises to maximize biosignal recording characteristics may be fabricated by manufacturing an elastic and air permeable substrate and combining the substrate with the manufactured microneedle to manufacture a microneedle patch.

### DESCRIPTION OF DRAWINGS

FIG. 1 is a flow chart of a method for manufacturing a polymer-based microneedle patch of the present invention.

FIG. 2 shows a microneedle array forming step of the present invention.

FIG. 3 shows a microneedle mold forming method of the present invention.

FIGS. 4A and 4B show a microneedle structure fabrication step of the present invention.

FIGS. 5A to 5C show microneedle tip shapes depending on change in a tilt angle (α) using a 3D printer of the present invention.

FIGS. 6A to 6C show forms depending on change in a tilt angle (α) of a microneedle structure tip fabricated by a microneedle mold of the present invention.

FIGS. 7A to 7C are images of microneedle made of polyimide of the present invention.

FIGS. 8A to 8C shows a microneedle patch manufacturing step of the present invention.

FIG. 9A to 9C are images of a shape memory polymer microneedle patch manufactured using the microneedle patch manufacturing step using the shape memory polymer of the present invention and the shape memory polymer.

FIG. 10 is a relationship graph between a 3D printing microneedle bevel angle (β) and a polyimide microneedle bevel angle (γ) depending on an adjustment of a 3D printing tilt angle (α) of the present invention.

FIG. 11 is a graph of skin insertion test results depending on a tilt angle (α) of the microneedle fabricated by the 3D printing tilt angle (α) adjustment method of the present invention.

FIG. 12 is a middle cross section of a polyimide microneedle having various forms of the present invention.

FIGS. 13A to 13C are images of a shape memory polymer microneedle fabricated by applying a shape memory polymer (SMP) and a shape memory polymer microneedle electrode completing metal deposition.

### BEST MODE

A method for manufacturing a polymer-based microneedle patch according to the present invention may include: microneedle structure fabrication of fabricating a structure in which a plurality of microneedles are arranged, using 3D printing; microneedle mold forming of adding the microneedle structure to a container containing a first polymer material to form a microneedle mold; microneedle array forming of injecting a second polymer material to the microneedle mold and performing curing to form a microneedle array; and microneedle patch manufacturing of arranging the microneedle array on a substrate to manufacture a microneedle patch.

In addition, the microneedle structure is formed by including a base part and a plurality of microneedles protruding from the base part, and in the microneedle structure fabrication, the base part may be placed at a certain tilt angle to a stage of a 3D printer.

Herein, the tilt angle at which the base of the microneedle structure is placed with the stage of the 3D printer may be 30 to 60°.

In addition, the middle section of the microneedle may have one of circular, triangular, tetragonal, hexagonal, or hexagonal star shape.

In addition, the first polymer material may be any one selected from a silicon polymer or a polyurethane, and the second polymer material may be a polyimide polymer or a shape memory polymer.

In addition, the microneedle patch manufacturing may include: coating solution preparation of preparing a coating solution; coating solution coating of coating a substrate surface with the prepared coating solution; metal pattern forming of forming a metal pattern on the substrate surface coated with the coating solution; microneedle array arranging of arranging the microneedle array on a metal pattern formed on the substrate surface; microneedle forming of forming a microneedle by removing the microneedle array pattern arranged on the metal pattern; and coating of coating the substrate and the metal pattern of the substrate with a third polymer material.

In addition, in the coating solution preparation, the coating solution may be prepared by selecting any one selected from a silicone rubber or a silicone resin, and mixing it with an organic solvent at a certain ratio and performing evaporation at a certain temperature.

In addition, in the coating solution coating, the substrate coated with the coating solution may be formed of a transparent plastic material.

In addition, in the coating solution coating, the third polymer material may be any one selected from a silicone rubber or a silicone resin.

In addition, the microneedle array forming may include: second polymer material injection of injecting a second polymer material into a microneedle mold in which grooves having the same incised shape as the microneedle structure are formed; vacuum placement of placing the mold to which the second polymer material has been injected under vacuum; microneedle array curing of curing the second polymer material injected into the microneedle mold placed under vacuum; and metal electrode coating of separating the cured microneedle array from the microneedle mold into which the second polymer material has been injected and coating the surface of the separated microneedle array with a metal electrode.

Next, a microneedle patch manufactured by the method for manufacturing a microneedle patch according to the present invention is provided, wherein a microneedle tip of the microneedle array may have an asymmetric structure having a 45 to 55° slope.

Next, a method for manufacturing a microneedle structure using printing is provided, wherein the microneedle structure is formed by including a base part and a plurality of microneedles protruding from the base part, and the base part may be placed at a tilt angle of 30 to 60° to a stage of a 3D printer.

### Mode for Carrying Out the Invention

Hereinafter, a technical idea of the present invention will be described in more detail with reference to the accompanying drawings. Prior to that, terms and words used in the present specification and claims are not to be construed as having a general or dictionary meaning but are to be construed as having meaning and concepts meeting the technical ideas of the present invention, based on a principle that the inventors may appropriately define the concepts of terms in order to describe their own inventions in best mode.

Therefore, since the configurations described in the exemplary embodiments and drawings of the present specification are merely most preferable exemplary embodiment but do not represent all of the technical spirit of the present invention, it should be understood that there may be various variant examples for replacing the above configurations at the time of filing this application.

Conventionally, fabrication of a microneedle using 3D printing technology has a problem in that forms of an actual designed model and a structure printed with a 3D printer are different, due to the resolution limit of the 3D printer. Therefore, in the present invention, in order to control the tilt angle of the microneedle so that it has durability and penetration power to a microneedle tip portion, a mold having the same shape as the structure of the microneedle printed using a 3D printer is manufactured, and a polymer is injected into the manufactured mold to fabricate a microneedle having excellent skin penetration and durability. Herein, the microneedle bevel refers to a tip at the very end which first comes into contact with the skin and is inserted into the skin when the microneedle is applied to the skin. In addition, an elastic and air permeable substrate is fabricated and the substrate is combined with the manufactured microneedle to manufacture a microneedle patch.

Hereinafter, a method for manufacturing a polymer-based microneedle patch will be described in detail with reference to the attached drawings.

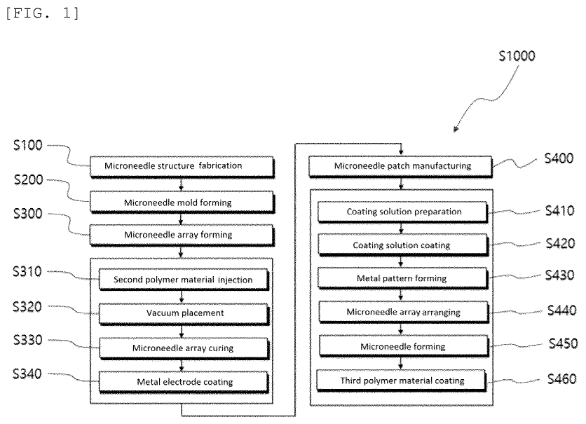

FIG. 1 is a flow chart of a method for manufacturing a polymer-based microneedle patch of the present invention. As shown in FIG. 1, the flow chart S1000 of the method for manufacturing a polymer-based microneedle patch of the present invention may include a microneedle structure fabrication step (S100), a microneedle mold forming step (S200), a microneedle array forming step (S300), and a microneedle patch manufacturing step (S400).

In the microneedle structure fabrication step (S100), a microneedle structure in which a plurality of microneedles are arranged may be fabricated using 3D printing. The microneedle structure may be formed by including a base part and a plurality of microneedles protruding from the base part. Specifically, in the microneedle structure fabrication step (S100), the base part may be placed at a certain tilt angle to the stage of a 3D printer. More specifically, the microneedle structure may be formed by including the base part and a plurality of microneedles protruding from the base part, and the base part may be placed at a tilt angle of 30 to 60° to the stage of the 3D printer. Since the manufacture of the microneedle array using a 3D printing process has a problem of a low resolution of a 3D printer, there is a limitation in manufacturing a precise microneedle array, and thus, a microneedle structure having an adjusted tilt angle may be manufactured using a 3D printer in the present invention.

In addition, in the microneedle mold forming step (S200), the microneedle structure may be added to a container containing the first polymer material to form a microneedle mold. The shape of the microneedle array may be manufactured more precisely by manufacturing the microneedle mold having the same incised shape as the microstructure. Herein, the first polymer material may be any one selected from a silicon-based polymer or a polyurethane, and more specifically, may be polydimethylsiloxane (PDMS).

In addition, the microneedle array forming step (S300) may further include: a second polymer material injection step (S310), a vacuum placement step (S320), a curing step (S330), and a metal electrode coating step (S340). The second polymer material may be injected into the microneedle mold and cured to form the microneedle array. The second polymer material is injected into the microneedle mold having the same incised shape as the microstructure to form the microneedle array, thereby manufacturing the microneedle array having a sharply formed tip portion. Herein, the second polymer material may be any one selected from a polyimide polymer or a shape memory polymer.

In addition, the microneedle patch manufacturing step (S400) may further include a coating solution preparation step (S410), a coating solution coating step (S420), a metal pattern forming step (S430), a microneedle array arranging step (S440), a microneedle forming step (S450), and a third polymer material coating step (S460). The microneedle array formed above may be attached to a substrate made of the third polymer material to manufacture a microneedle patch. The microneedle array manufactured above is arranged in the microneedle patch, thereby manufacturing a microneedle patch having improved penetration power and flexibility simultaneously. Herein, the microneedle tip of the microneedle array manufactured above may be fabricated to have an asymmetric structure of 45 to 55°.

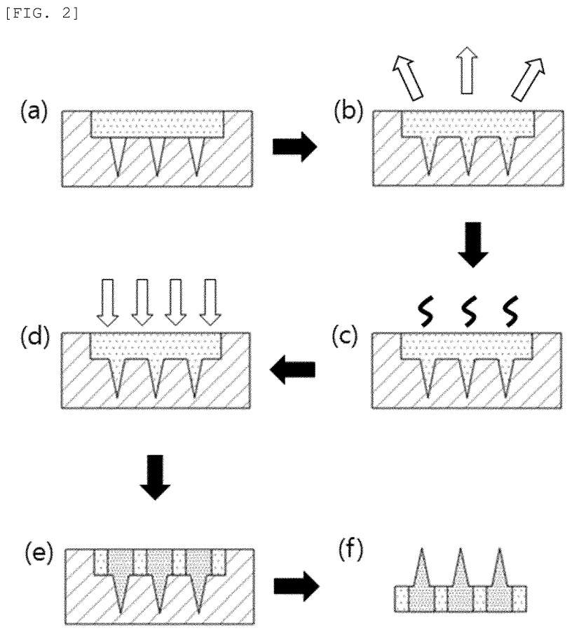

FIG. 2 shows a microneedle array forming step of the present invention. The microneedle array forming step (S300) may include: a second polymer material injection step (S310), a vacuum placement step (S320), a microneedle array curing step (S330), and a metal electrode coating step (S340).

As shown in (a) of FIG. 2, the second polymer material injection step (S310) may be a step of injecting the second polymer material into a microneedle mold in which grooves having the same incised shape as the microneedle structure are formed. Herein, the second polymer material may be any one selected from a polyimide polymer or a shape memory polymer.

In addition, as shown in (b) of FIG. 2, the vacuum placement step (S320) may be a step of placing the mold into which the second polymer material has been injected under vacuum for about 30 minutes. Herein, since there is an effect of easily filling the end part of the mold with the second material without external pressure by placing the mold into which the second material has been injected under vacuum, a microneedle having the same shape as the microneedle structure may be manufactured. In addition, the microneedle array curing step (S330) may further include curing the mold placed under vacuum. Herein, the microneedle array curing step (S330) may be first curing of curing by placing in an oven as shown in (c) of FIG. 2, second curing of curing by irradiation with ultraviolet rays as shown in (d) of FIG. 2, and third curing of curing by placing in an oven as shown in (e) of FIG. 2. The first curing may be performed in an oven at 120° C. for 10 minutes. In addition, the second curing may be performed better by irradiation with ultraviolet rays for 1 hour, and the third curing may be performed in an oven at 200° C. for 1 hour. Therefore, the mold into which the second material has been injected undergoes the first curing, the second curing, and the third curing, so that the second material may be completely cured, and thus, the second material is completely separated from the mold into which the second material has been injected to fabricate a microneedle having the same shape as the mold. Herein, the microneedle of the microneedle array manufactured above may have an aspect ratio of 2.5:1 to 3.5:1.

In addition, as shown in (f) of FIG. 2, the metal electrode coating step (S340) may be a step of coating a metal electrode on the surface of the microneedle array made of the second polymer material. Herein, the metal electrode may be coated with any one of Cr/Au or Ti/TiN/Mo, using a sputtering process. Herein, the surface of the microneedle array is coated with the metal electrode, thereby fabricating a microneedle which may be used as a bioelectrode.

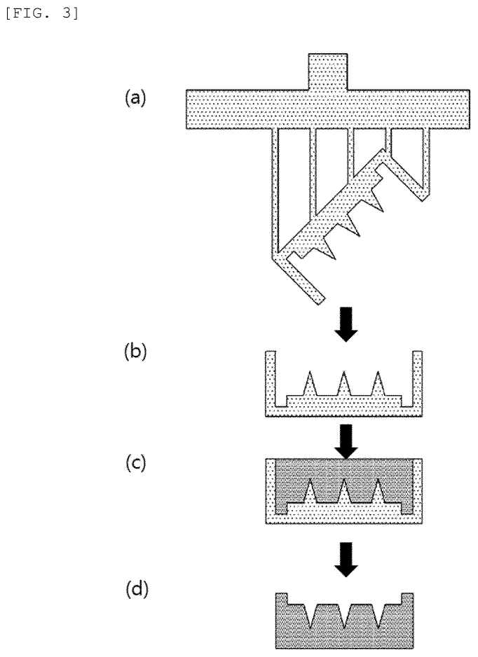

FIG. 3 shows the microneedle mold forming method of the present invention. As shown in FIG. 3, the microneedle mold forming step (S200) may further include a second polymer material injection step and a microneedle mold manufacturing step. (a) of FIG. 3 shows a microneedle mold printed with a 3D printer. A support is removed from the microneedle mold printed with the 3D printer of (a) of FIG. 3 to manufacture a microneedle structure manufactured by the 3D printer of (b) of FIG. 3. In addition, as shown in (c) of FIG. 3, the microneedle structure adding step may be a step of adding the microneedle structure to a container containing the first polymer material so as to have the same incised shape as the microneedle structure of (b) of FIG. 3. Herein, the first polymer material may be any one selected from a silicon-based polymer or a polyurethane, and more specifically, may be polydimethylsiloxane (PDMS).

In addition, as shown in (d) of FIG. 3, in the microneedle mold manufacturing step, a mold in which grooves having the same incised shape as the added microneedle structure are formed may be manufactured.

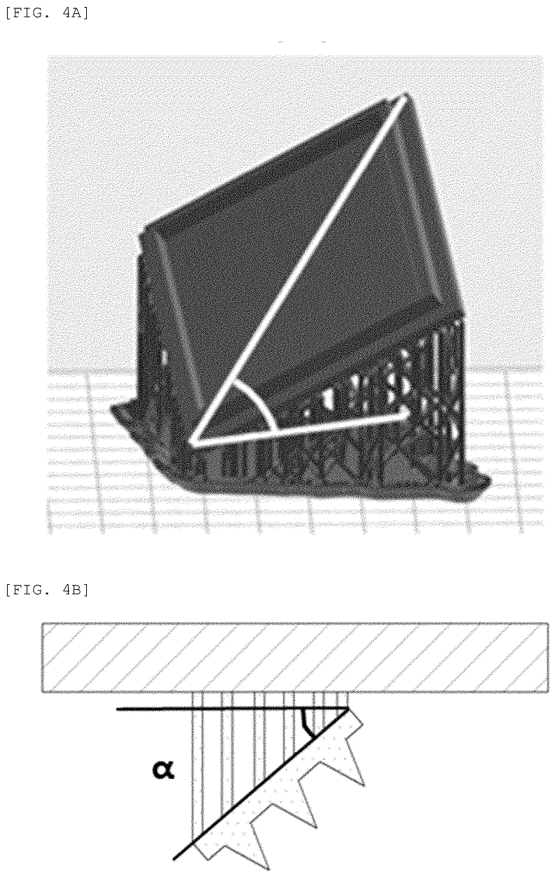

FIGS. 4A and 4B show a microneedle structure fabrication step of the present invention. As shown in FIGS. 4A and 4B, the microneedle structure fabrication step (S100) may further include a 3D modeling step and a microneedle structure printing step. As shown in FIG. 4A, in the setting of the tilt angle of the microneedle, a support is modeled together during 3D modeling so that the microneedle structure has a desired tilt angle and the tilted angle is formed in the structure when the structure is printed with a 3D printer, or the tilt angle of the modeling stage of the 3D printer is set so that the microneedle structure has a desired tilt angle and the microneedle having the desired tilt angle may be printed. A microneedle structure having the same shape as a microneedle to be fabricated may be printed by printing the microneedle structure by a 3D printer. In addition, as shown in (b) of FIG. 4, the 3D modeling step may be a step of 3D modeling so that the shape of the microneedle has a tilt angle (α). When the microneedle structure is manufactured by the 3D printer, it is difficult to manufacture a microneedle structure having a precise tip angle due to the resolution of the 3D printer, and thus, the 3D modeling is performed so that the shape of the microneedle has the tilt angle (α) in the 3D modeling step, thereby manufacturing the angle of the microneedle tip more precisely.

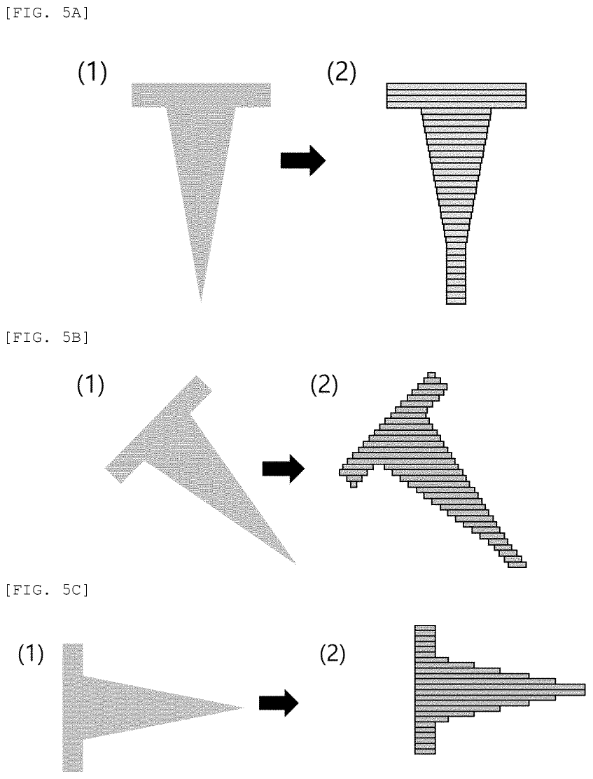

FIGS. 5A to 5C show microneedle tip shapes depending on change in a tilt angle (α) of the microneedle using a 3D printer of the present invention. As shown in FIGS. 5A to 5C, in order to overcome the resolution limit of the 3D printing, the tilt angle (α) of the 3D printer is set to fabricate the microneedle structure using the 3D printer.

FIGS. 5A to 5C show the microneedle structure fabricated using the 3D printer, and the microneedle structure was manufactured by setting the tilt angle (α) of the microneedle structure manufactured by the 3D printer to 0 to 90°. (1) of FIG. 5A is a microneedle modeling drawing which was modeled when the tilt angle of the microneedle structure was 0°, by 3D modeling, and (2) of FIG. 5A is a schematic diagram showing the microneedle structure manufactured by the 3D printer when the tilt angle (α) is 0°. Upon comparison with (1) of FIG. 5A, it was found that the tip of the microneedle structure was formed bluntly when the tilt angle (α) of the microneedle structure was 0°.

In addition, (1) of FIG. 5B is a microneedle modeling drawing which was modeled when the tilt angle was 45°, by 3D modeling, and (2) of FIG. 5B is a schematic diagram showing the microneedle structure manufactured by the 3D printer when the tilt angle (α) is 45°. Upon comparison with (1) of FIG. 5B, it was found that the tip of the microneedle structure was formed sharply when the tilt angle (α) of the microneedle structure was 45°.

In addition, (1) of FIG. 5C is a microneedle modeling drawing which was modeled when the tilt angle was 90°, by 3D modeling, and (2) of FIG. 5C is a schematic diagram showing the microneedle structure manufactured by the 3D printer when the tilt angle (α) is 90°. Upon comparison with (1) of FIG. 5C, it was found that the tip of the microneedle structure was formed bluntly when the tilt angle (α) of the microneedle structure was 90°.

Therefore, it was confirmed that the tip of the microneedle structure manufactured using the 3D printer was formed sharply when the tilt angle (α) of the microneedle structure was 45°, according to the principle of the 3D printing in which fabrication is performed layer by layer, as shown in FIG. 5.

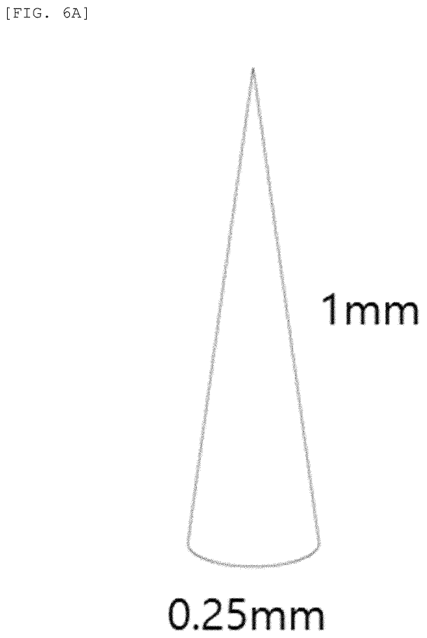

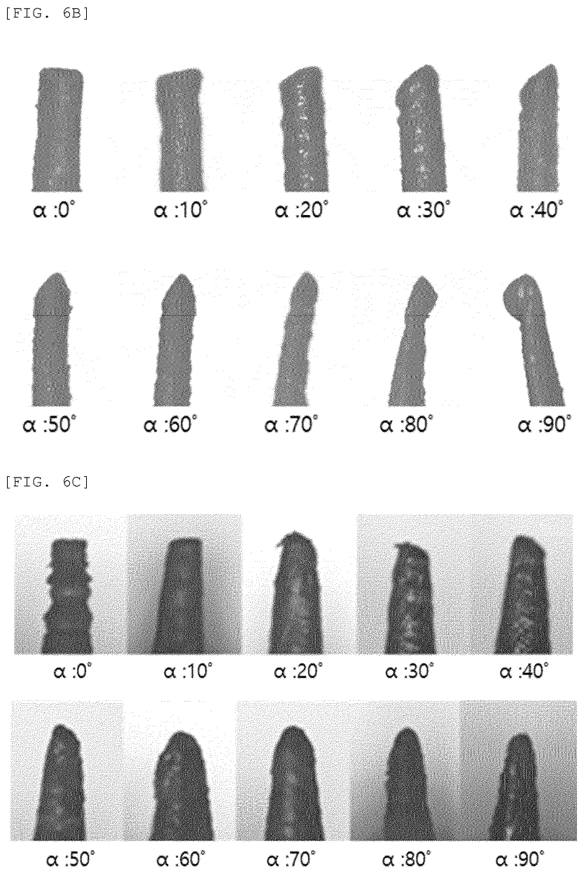

FIGS. 6A to 6C show forms depending on change in a tilt angle (α) of a microneedle structure tip fabricated by a microneedle mold of the present invention. FIG. 6A shows a 3D-modeled microneedle. As shown in FIG. 6A, the tip of the 3D-modeled microneedle may be formed sharply and designed, and the microneedle may have an aspect ratio of 1.5:1 to 5.5:1. FIG. 6B shows the shape of the microneedle tip depending on the tilt angle of the microneedle manufactured by fabricating the microneedle structure printed so that the microneedle has the tilt angle (α), using a 3D printer into a mold and injecting a polymer into a mold having the same shape of the microneedle structure. As shown in FIGS. 6B and 6C, it was confirmed that as the tilt angle of the microneedle manufactured by the 3D printer and the microneedle mold was increased, the tip of the microneedle became sharper. In addition, it was observed that the tip shape of the microneedle was bent from when the tilt angle (α) of the microneedle manufactured by the 3D printer and the microneedle mold was 60°, and this may be a phenomenon of bending due to the influence of gravity.

In addition, as shown in FIGS. 6B and 6C, it was found that the microneedle tip was formed sharply at the tilt angle (α) of the microneedle between 30° and 50°.

Therefore, it was found that the microneedle made of the microneedle structure printed by the 3D printer has a sharp tip when it has the tilt angle (α) of 30° to 50°. Specifically, the tip of the microneedle of the microneedle array may have an asymmetric structure having a slope of 45° to 55°.

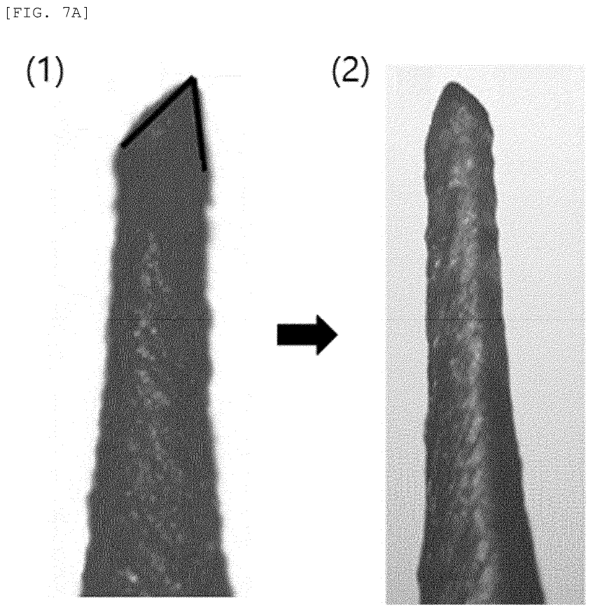

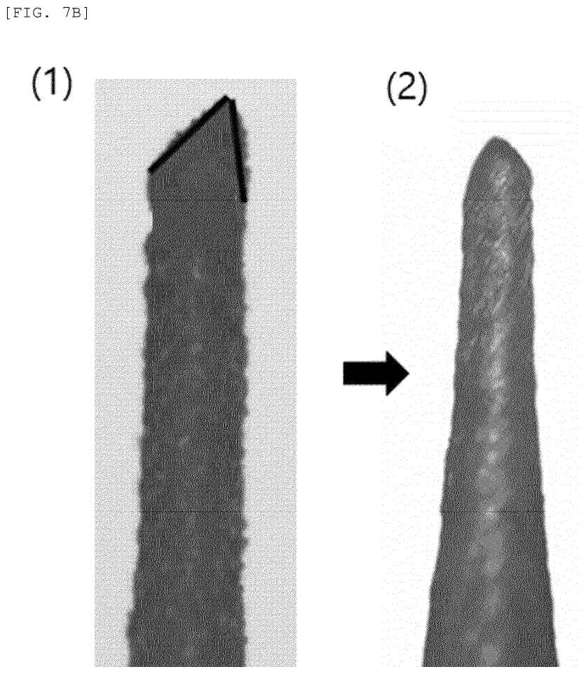

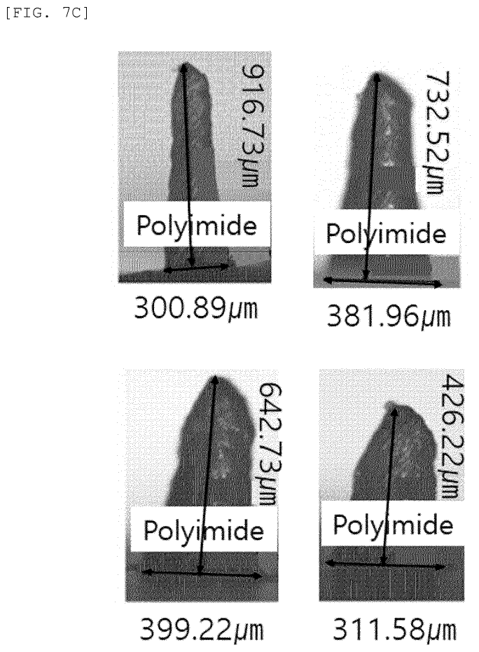

FIGS. 7A to 7C show images of the microneedle made of polyimide of the present invention. (1) of FIG. 7A shows a microneedle structure having the tilt angle (α) of 45° which was printed by the 3D printer, and (2) of FIG. 7A shows the shape of the tip portion of the microneedle array fabricated by injecting polyimide into the microneedle mold having the same shape as (1) of FIG. 7A. As shown in (1) and (2) of FIG. 7A, it was confirmed that the angle and the tilt angle (90−α=β=46.38°) of the microneedle structure manufactured by applying the method of adjusting the tilt angle of the microneedle structure using the 3D printer of the present invention and the molding method fabricated by the microneedle structure having the adjusted tilt angle and the microneedle array tip made of polyimide were almost the same.

(1) of FIG. 7B shows a microneedle structure having the tilt angle (α) of 40° which was printed by the 3D printer, and (2) of FIG. 7B shows the shape of the tip portion of the microneedle array fabricated by injecting polyimide into the microneedle mold having the same shape as (1) of FIG. 7B. As shown in (1) and (2) of FIG. 7B, it was confirmed that the angle and the tilt angle (90−α=β=50.84°) of the microneedle structure manufactured by applying the method of adjusting the tilt angle (α) of the microneedle structure using the 3D printer of the present invention and the molding method fabricated by the microneedle structure having the adjusted tilt angle and the microneedle array tip made of polyimide were almost the same.

In addition, FIG. 7C shows the shapes of the polyimide microneedle array tip portion having various lengths fabricated when a printing angle was 45°. As shown in FIG. 7C, it was found that the tilt angle (α) of the microneedle structure may be adjusted using the 3D printer of the present invention, and also the length of the microneedle may be adjusted. Thus, it was confirmed that the angle, the shape, and also the length of the microneedle tip may be adjusted by the microneedle structure having the adjusted tilt angle (α) printed with the 3D printer suggested in the present invention, the microneedle mold manufactured by the microneedle structure, and the microneedle array manufactured by the microneedle mold, and these may be reflected on the fabrication of the polyimide microneedle.

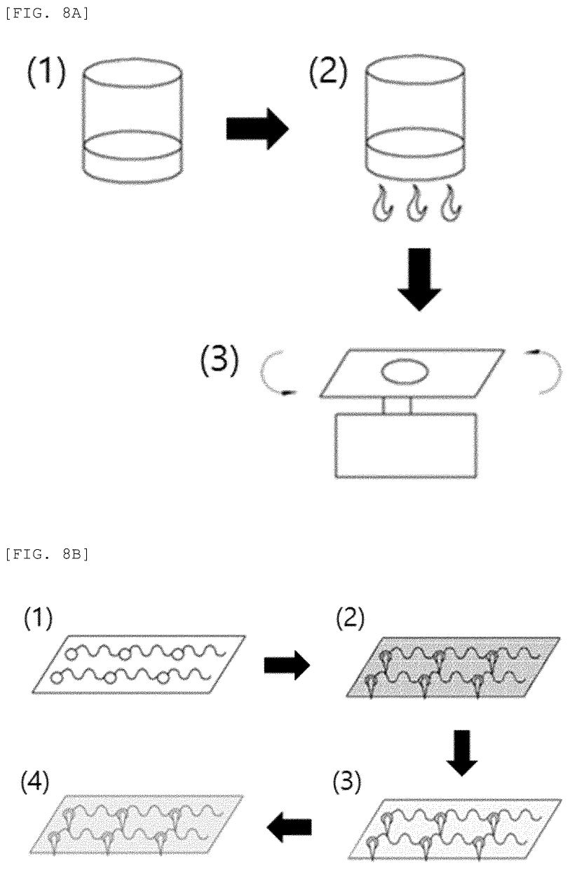

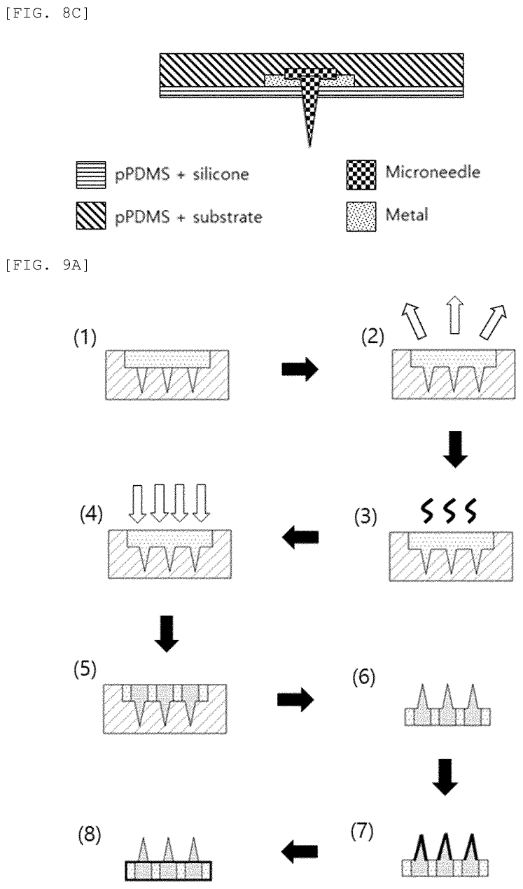

FIGS. 8A to 8C show the microneedle patch manufacturing step of the present invention. The microneedle patch manufacturing step (S400) may include a coating solution preparation step (S410), a coating solution coating step (S420), a metal pattern forming step (S430), a microneedle array arranging step (S440), a microneedle forming step (S450), and a third polymer material coating step (S460).

Herein, as shown in (1) of FIG. 8, in the coating solution preparation step (S410), the coating solution may be any one selected from a silicone rubber or a silicone resin. Specifically, it may be polydimethylsiloxane (PDMS). In addition, in the coating solution, the polydimethylsiloxane (PDMS) may be mixed with an organic solvent at a certain ratio. More specifically, a mixing ratio of polydimethylsiloxane (PDMS), toluene, citric acid, and ethanol may be 20:20:10:10.

In addition, as shown in (2) of FIG. 8A, the mixed coating solution may evaporate at a certain temperature. Specifically, the certain temperature may be 150° C. More specifically, the coating solution mixed with the organic solvent may evaporate at a certain temperature and be prepared into porous polydimethylsiloxane (pPDMS).

In addition, as shown in (3) of FIG. 8A, in the coating solution coating step (S420), a substrate surface may be coated with the p-polydimethylsiloxane coating solution prepared above by a spin coating process. In addition, the substrate coated with the p-polydimethylsiloxane coating solution may be made of a transparent plastic material. Herein, the plastic material may be polyethylene terephthalate (PET). In addition, herein, after coating with the p-polydimethylsiloxane coating solution, an insulating material may be further coated.

In addition, as shown in (1) of FIG. 8B, the metal pattern forming step (S430) may be a step of forming a metal pattern on the substrate coated with the coating solution. The metal pattern forming step (S430) may be performed by a photolithography process of semiconductor. More specifically, an etching process and a photoresist removing process may be applied for etching the metal pattern.

In addition, as shown in (2) of FIG. 8B, the microneedle array arranging step (S440) may be a step of arranging the microneedle array on the metal pattern formed on the substrate. The microarray arrangement may be the microarray attached by a silicone resin or an epoxy resin being arranged on the metal pattern formed on the substrate.

In addition, as shown in (3) of FIG. 8B, the microneedle forming step (S450) may be a step of forming a microneedle by removing the microneedle array pattern arranged on the metal pattern. After applying the developing solution on the microneedle array on the substrate and exposing it to a UV light source, an exposed area and an unexposed area are selectively removed to form a microneedle, and thus, the microneedle patch formed of the microneedle separated from the microneedle array may be finally manufactured.

In addition, as shown in (4) of FIG. 8B, the third polymer material coating step (S460) may be a step of coating the third polymer material on the substrate and on the metal pattern of the substrate. Herein, the third polymer material may be any one selected from a silicone rubber and a silicone resin. More specifically, the third polymer material may be a mixture of p-polydimethylsiloxane (pPDMS) and silicon. The substrate and the metal pattern of the substrate are coated with the third polymer material, so that it may act as an insulating layer which minimizes capacitance and electrical interference. FIG. 8C shows a cross-sectional schematic diagram of the microneedle patch of the present invention. The microneedle patch may be fabricated by the manufacturing processes of FIGS. 8A and 8B. The microneedle patch formed by the manufacturing processes of FIGS. 8A and 8B is described again, as follows. The microneedle array is arranged on the metal pattern formed on the substrate coated with p-polydimethylsiloxane (pPDMS), and the microneedle array pattern arranged on the metal pattern is removed to form the microneedle. The substrate and the metal pattern on the substrate are coated with the third polymer material which is a mixture of p-polydimethylsiloxane (pPDMS) and silicon, thereby finally fabricating the microneedle patch as in FIG. 8C.

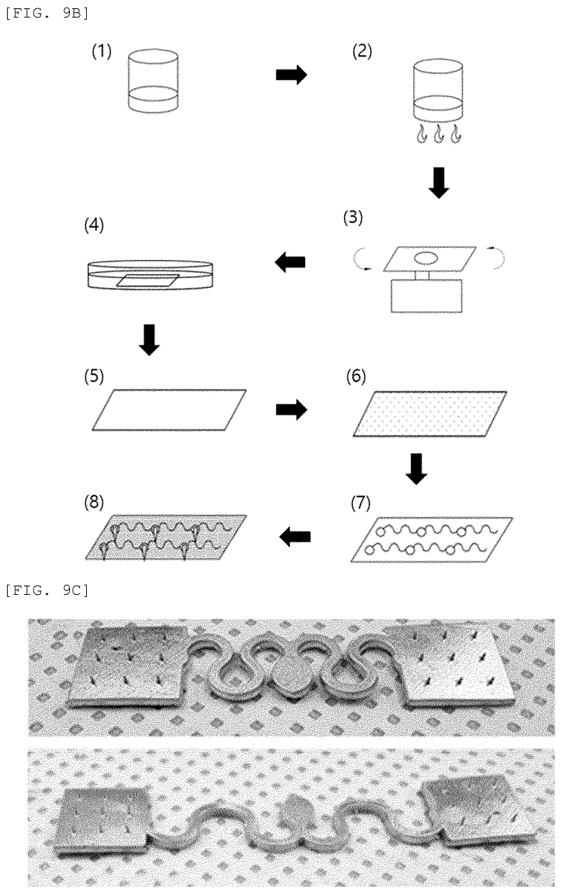

FIG. 9A to 9C are images of a shape memory polymer microneedle patch manufactured using the microneedle patch manufacturing step using the shape memory polymer of the present invention and the shape memory polymer. FIG. 9A shows a microneedle manufacturing step using a shape memory polymer (SMP) of the present invention. The method for manufacturing a microneedle patch using the shape memory polymer may be performed by applying a process other than the microneedle patch manufacturing step shown in FIGS. 8A to 8C, for process efficiency. In the shape memory polymer microneedle patch, a cap was put on the microneedle electrode portion for parylene-C coating on a part other than the microneedle electrode portion, as shown in (7) of FIG. 9A, parylene-C coating was performed, and then the cap was removed, thereby further forming an insulating layer in the portion other than the microneedle electrode, as shown in (8) of FIG. 9A. FIG. 9B shows a microneedle patch manufacturing step using a shape memory polymer (SMP) of the present invention. The microneedle patch using the shape memory polymer (SMP) further includes a process of immersing in ethanol for removing a citric acid crystal, as shown in (4) of FIG. 9B. In addition, a process of drying and coating a silicon adhesive (Silbione) after the immersion may be further included, as shown in (5) and (6) of FIG. 9B. In addition, FIG. 9C is a shape memory polymer microneedle patch manufactured by the method of FIGS. 9A and 9B. Referring to FIG. 9A, the microneedle manufacturing step using the shape memory polymer (SMP) of the present invention will be described. Referring to (1) and (2) of FIG. 9A, the second polymer may be injected into the microneedle mold in which grooves having the same incised shape as the microneedle structure are formed. In addition, (3) of FIG. 9A shows that the mold into which the second polymer material has been injected is placed under vacuum for about 30 minutes. By placing the mold into which the second polymer material has been injected under vacuum, the microneedle having the same shape as the microneedle structure may be manufactured. Next, (4) of FIG. 9A shows a process of irradiating with ultraviolet rays and curing, and (5) of FIG. 9A shows a process of curing at 200° C. for 1 hour. (6) of FIG. 9A shows that the surface of the microneedle cured at 200° C. for 1 hour is coated with a Cr/Au metal by a sputtering process to form a metal electrode, and then is subjected to a wiring process. (7) of FIG. 9A shows that the surface of the microneedle coated with the metal electrode is coated with parylene-C to finally manufacture a microneedle using the shape memory polymer (SMP). In addition, (8) of FIG. 9A shows that a cap was put on the microneedle electrode, parylene-C coating was performed, and then the cap was removed to further form an insulating layer on a portion other than the microneedle electrode.

Next, referring to FIG. 9B, the microneedle patch manufacturing step using the shape memory polymer (SMP) of the present invention will be described.

(1) of FIG. 9B shows the coating solution preparation step, and the coating solution may be prepared by mixing polydimethylsiloxane (PDMS), toluene, citric acid, and ethanol at a certain ratio of 20:20:10:10. In addition, (2) of FIG. 9B shows a process of evaporating the mixed coating solution at a certain temperature of 150° C. to prepare porous Polydimethylsiloxane (pPDMS). (3) of FIG. 9B shows that the substrate surface is coated with the prepared porous Polydimethylsiloxane (pPDMS) coating solution by a spin coating process, and the substrate coated with the p-polydimethylsiloxane coating solution may be made of a transparent plastic material of polyethylene terephthalate (PET). In addition, as shown in (4) of FIG. 9B, the substrate coated with the p-polydimethylsiloxane coating solution is subjected to a process of being immersed in ethanol for removing a citric acid crystal and then a drying process, as shown in (5) of FIG. 9B, and coated with a silicon adhesive (Silbione), as shown in (6) of FIG. 9B. In addition, as shown in (7) and (8) of FIG. 9B, a metal pattern is formed on the substrate which is coated with the coating solution after coated with a silicon adhesive (Silbione), and may be arranged on the metal pattern in which the microarray is adhered by a silicone resin or an epoxy resin to be formed on the substrate.

Therefore, the shape memory polymer microneedle patch as shown in FIG. 9C may be manufactured after undergoing the processes of FIGS. 9A and 9B.

FIG. 10 shows a relationship between a 3D printing microneedle bevel angle (β) and a polyimide microneedle bevel angle (γ) depending on adjustment of a 3D printing tilt angle (α) of the present invention, as a graph. Referring to FIG. 10, similarity is shown between the bevel angle (β) of the 3D printed microneedle and the bevel angle (γ) of the polyimide microneedle. In addition, it was found that when the 3D printing tilt angle (α) was 40°, the bevel angle (β) was the lowest.

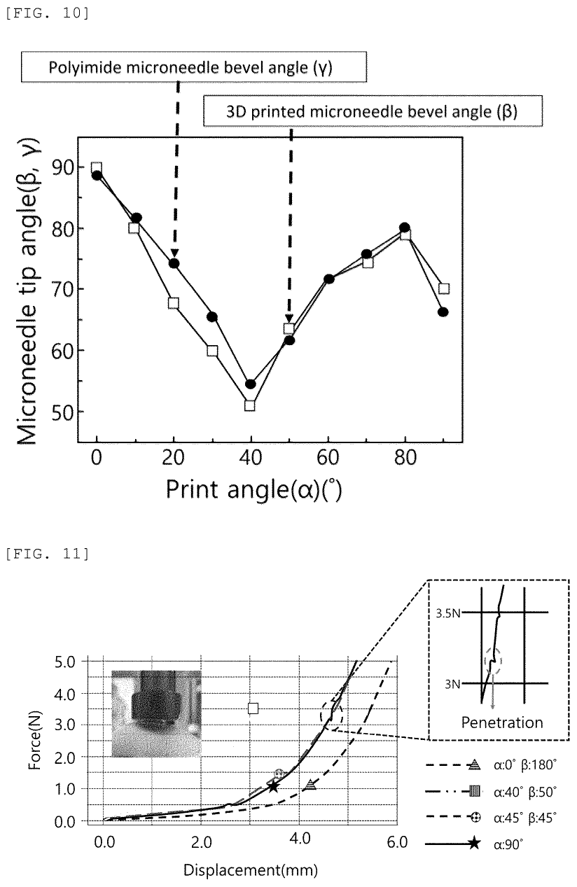

FIG. 11 is a graph of skin insertion test results depending on a tilt angle (α) of the microneedle fabricated by the 3D printing tilt angle (α) adjustment method of the present invention. When the test was performed by inserting the polyimide microneedle fabricated according to the present invention into the pig skin as a subject, a tendency for pressure applied to the microneedle to decrease is shown as the angle of the tip of the microneedle fabricated by a 3D printer and a molding process increases. It was found therefrom that as the angle of the microneedle tip increases, the durability of the microneedle is improved. Specifically, it was found that a penetration phenomenon of going in and popping out occurred in the graph of the polyimide microneedle (β=50.84°) fabricated when the tilt angle (α) of the microneedle fabricated using the 3D printer and the molding process was 40°. This means that the microneedle penetrated the skin, and it was found that the microneedle at the other tilt angles (α) was pushed without penetrating the skin. Therefore, only the polyimide microneedle (β=50.84°) fabricated when the microneedle tilt angle (α) was 40° penetrated the pig skin, and thus, the microneedle made of polyimide when the microneedle tilt angle (α) was 40° had an effect on skin penetration performance.

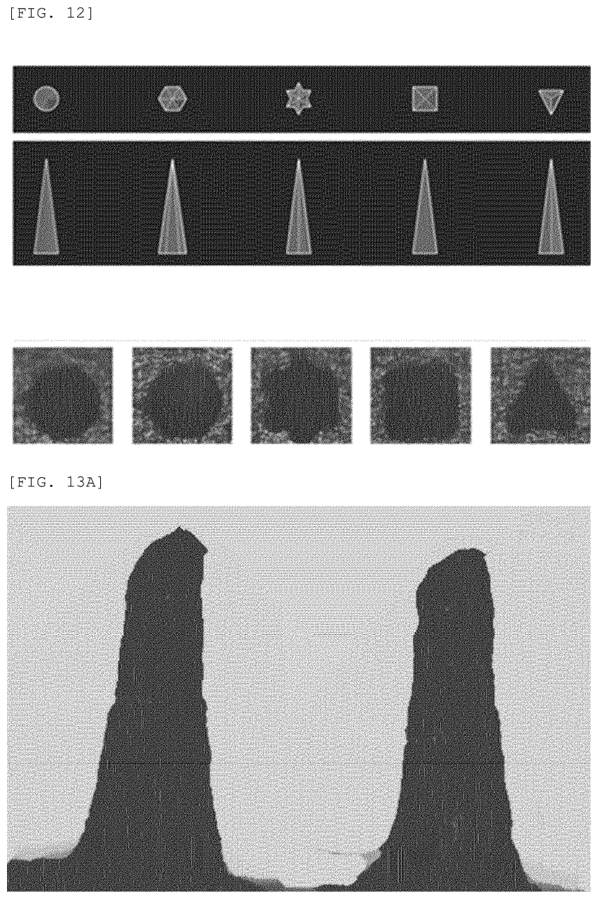

FIG. 12 is a middle cross section of a polyimide microneedle having various forms of the present invention. As shown in FIG. 12, the middle cross section of the microneedle having various forms may be designed and fabricated. Specifically, the middle cross section of the microneedle may have any one of circular, triangular, tetragonal, hexagonal, or hexagonal star shape, and the microneedle may be fabricated into various forms, without being limited thereto. Therefore, the microneedle array having various forms may be designed by a molding process in which the microneedle structure having a tilt angle adjusted by a 3D printer and the above microneedle structure have the same shape, and thus, the function of the microneedle such as flexibility and rigidity may be optimized.

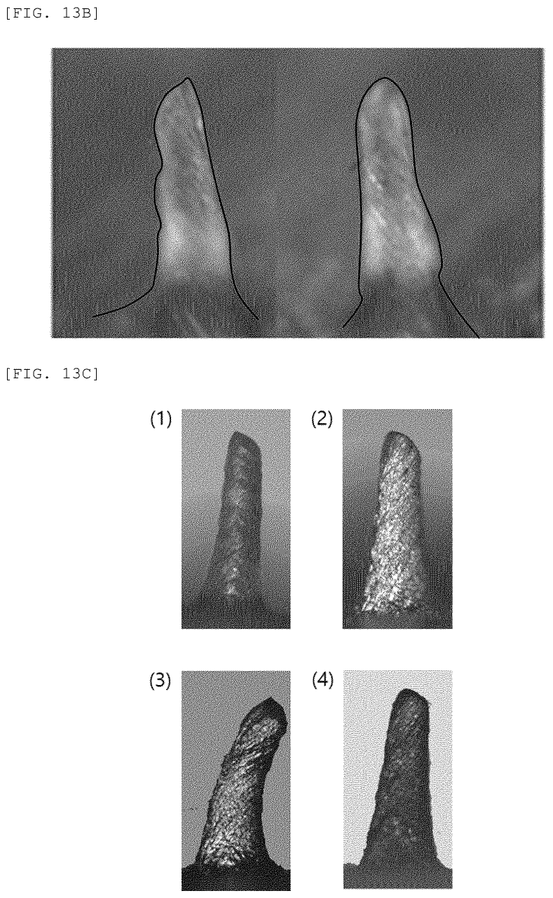

FIGS. 13A to 13C are images of a shape memory polymer microneedle fabricated by applying a shape memory polymer (SMP) and a shape memory polymer microneedle electrode completing metal deposition. FIG. 13A shows the shape of the tip portion of the microneedle array fabricated by injecting polyimide into a microneedle mold having the same shape as the microneedle structure printed by the 3D printer. FIG. 13B is an actual photograph of the shape memory polymer microneedle fabricated by the method of FIG. 13A. (1) of FIG. 13C shows the appearance before the sputtering process of the shape memory polymer microneedle fabricated by the method of FIG. 13A. After the Cr/Au sputtering process, the shape memory polymer microneedle having the metal thin film formed as shown in (2) of FIG. 13C may be fabricated. (3) of FIG. 13C shows the shape memory polymer microneedle after repeating the skin insertion test 10 times, in the same manner as in FIG. 10A. Referring to (3) of FIG. 13C, the results in which the shape memory polymer microneedle was bent due to the repeated insertion may be seen. (4) of FIG. 13C shows the results of heating the bent shape memory polymer microneedle of (3) of FIG. 13C at 45° C. for 1 minute. Referring to (4) of FIG. 13C, it was confirmed that the bent shape memory polymer microneedle was restored to its original form after heating it at 45° C. for 1 minute. Therefore, as shown in FIG. 13C, to the microneedle of the present invention, various biocompatible polymers and functional polymers may be applied, without limiting the applicable materials to polyimide. The shape memory polymer may be implemented into a desired shape depending on temperature change, and is softened as the temperature rises. Therefore, the flexibility and rigidity of the microneedle may be maximized by using the functional polymer such as the shape memory polymer.

In addition, the fabricated polymer microneedle may be combined with a polydimethylsiloxane (PDMS) substrate which is a polymer-based substrate having high air permeability by depositing metal on the surface of the polymer microneedle, and may be used as an elastic and air permeable electromyography microelectrode with minimum invasion.

The present invention is not limited to the above-mentioned exemplary embodiments, and may be variously applied, and may be variously modified without departing from the gist of the present invention claimed in the claims.

### INDUSTRIAL APPLICABILITY

The present invention relates to a method for manufacturing a polymer-based microneedle patch and a microneedle patch manufactured therefrom, and biosignal recording characteristics may be maximized by allowing insertion with minimum invasion using the method for manufacturing a polymer-based microneedle patch and the microneedle patch manufactured therefrom, and minimizing skin tissue damage and noises by flexible polymer properties after the insertion.

1. A method for manufacturing a polymer-based microneedle patch, the method comprising:
microneedle structure fabrication of fabricating a structure in which a plurality of microneedles are arranged, using 3D printing;
microneedle mold forming of adding the microneedle structure to a container containing a first polymer material to form a microneedle mold;
microneedle array forming of injecting a second polymer material to the microneedle mold and performing curing to form a microneedle array; and
microneedle patch manufacturing of arranging the microneedle array on a substrate to manufacture a microneedle patch.

2. The method for manufacturing a polymer-based microneedle patch of claim 1,
wherein the microneedle structure is formed by including a base part and a plurality of microneedles protruding from the base part, and
in the microneedle structure fabrication, the base part is placed at a certain tilt angle to a stage of a 3D printer.

3. The method for manufacturing a polymer-based microneedle patch of claim 2, wherein the tilt angle at which the base of the microneedle structure is placed with the stage of the 3D printer is 30 to 60°.

4. The method for manufacturing a polymer-based microneedle patch of claim 2, wherein a middle cross section of the microneedle has one of circular, triangular, tetragonal, hexagonal, or hexagonal star shape.

5. The method for manufacturing a polymer-based microneedle patch of claim 1,
wherein the first polymer material is any one selected from a silicon-based polymer or polyurethane, and
the second polymer material is a polyimide polymer or a shape memory polymer.

6. The method for manufacturing a polymer-based microneedle patch of claim 1, wherein the microneedle patch manufacturing includes:
coating solution preparation of preparing a coating solution;
coating solution coating of coating a substrate surface with the coating solution prepared above;
metal pattern forming of forming a metal pattern on the substrate surface coated with the coating solution;
microneedle array arranging of arranging the microneedle array on the metal pattern formed on the substrate surface;
microneedle forming of removing the microneedle array pattern arranged on the metal pattern to form a microneedle; and
third polymer material coating of coating the substrate and the metal pattern on the substrate with a third polymer material.

7. The method for manufacturing a polymer-based microneedle patch of claim 6, wherein the coating solution is any one selected from a silicone rubber and a silicone resin, and is prepared by mixing it with an organic solvent at a certain ratio and performing evaporation at a certain temperature.

8. The method for manufacturing a polymer-based microneedle patch of claim 6, wherein the substrate coated with the coating solution in the coating solution coating is formed of a transparent plastic material.

9. The method for manufacturing a polymer-based microneedle patch of claim 6, wherein in the coating solution coating, the third polymer material is any one selected from a silicone rubber or a silicone resin.

10. The method for manufacturing a polymer-based microneedle patch of claim 1, wherein the microneedle array forming includes:
second polymer material injection of injecting the second polymer material into the microneedle mold in which grooves having the same incised shape as the microneedle structure are formed;
vacuum placement of placing the mold into which the second polymer material has been injected under vacuum;
microneedle array curing of curing the second polymer material injected into the microneedle mold placed under vacuum; and
metal electrode coating of separating the cured microneedle array from the microneedle mold into which the second polymer material has been injected and coating the surface of the separated microneedle array with a metal electrode.

11. A polymer-based microneedle patch manufactured by the method for manufacturing a microneedle patch of claim 1, wherein a microneedle tip of the microneedle array has an asymmetric structure having a slope of 45 to 55°.

12. A method for manufacturing a microneedle structure using 3D printing,
wherein the microneedle structure is formed by including a base part and a plurality of microneedles protruding from the base part, and
the base part is placed at a tilt angle of 30 to 60° to a stage of a 3D printer.
